(19) United States
(12) Patent Application Publication
(10) Pub. No.: US 2023/0410979 A1
(43) Pub. Date: Dec. 21, 2023

(54) LOCATION-BASED MULTI-USER AUGMENTED REALITY FOR WELLNESS

(71) Applicant: TRIPP, Inc., Los Angeles, CA (US)

(72) Inventors: Nanea Reeves, Sherman Oaks, CA (US); Jason Lee Asbahr, Oakland, CA (US); Felipe Lara, Los Angeles, CA (US); Wilson O'neal Westbrook-Fergeson, Oakland, CA (US); Zachary Clark Krausnick, Ashland, OR (US); Matthew Francis Bracks, Glendale, CA (US); Adrian Mark Ludley, Palo Alto, CA (US); Daniel Abram Kharlas, London (CA)

(21) Appl. No.: 17/845,979

(22) Filed: Jun. 21, 2022

(51) Int. Cl. G16H 20/70; A61B 5/0205; A61B 5/11; A61B 5/00

(52) U.S. Cl. CPC G16H 20/70; A61B 5/0205; A61B 5/1112; A61B 5/744; A61B 5/024

(57) ABSTRACT

An augmented wellness environment is generated at a media processing device, where the augmented wellness environment includes a virtual object. The content of the augmented wellness environment is determined by the activities (e.g., participation in wellness activities) of the user and one or more additional users (e.g., users at a similar location to the user). The augmented wellness environment may be modified based on interactions between users, and the augmented wellness environment may also be modified based on user activity data, such as biometric data (e.g., heart rate, breathing rate, etc.) of one or more users experiencing the augmented wellness environment. Using the user activity data, the system may determine a user's present mental state. The system can modify the augmented wellness environment to alter the user's present mental state to a target mental state.

### BACKGROUND

### Technical Field

This disclosure relates generally to a media content system, and, more specifically, to a media content system that provides an augmented wellness experience based on the interactions of users.

### Description of the Related Art

Conventional media content systems are typically capable of providing static content such as movies or interactive content (such as video games) that may respond to actively controlled inputs provided by a user. For wellness applications (e.g., guided meditation, relaxation, focus activities, or other mood improvement applications), such media content systems have limited effectiveness because they neglect other users when generating content for one user, isolating the user in their own experience. In augmented reality (AR) applications, virtual content is overlaid on either a video feed depicting the user's environment (e.g., on the screen of a smart phone) or the user's environment itself (e.g., using a head mounted display such as AR glasses). An AR experience may be customized to the user's preferences, but the user's experience is typically disconnected from that of other users. Specifically, because users' experiences may be individually customized, different users in the same location at the same time may have very different experiences, potentially resulting in the experiences isolating users, causing them to focus on their own “world.” This is undesirable for wellness applications that should connect users in a community that supports and fosters a collective wellness to truly promote wellbeing.

### SUMMARY

A media system uses location data and activity information of users to alter a mental state of a user. The media system augments audio or visual elements of a digitally-rendered wellness application using a user's location and the interactions between the user and at least one other user. The media system uses sensors to track the user's location and measure user activity (e.g., biometric activity such as heart rate) of the user. Using the sensed information, the media system may determine the user's present mental state. The media system can modify the digitally-rendered wellness application to alter the user's present mental state to a target mental state. In one example, the measured biometric activity may indicate that a user in a group meditation activity is experiencing an increased heart rate associated with anxiety. The media system can determine to modify the ambient music output for the users participating in the group meditation activity based on a collective mental state that accounts for the users' heart rates, including the elevated heart rate of the user who may be anxious. Accordingly, the media system generates a virtual experience (e.g., AR or virtual reality (VR) experience) that can dynamically change as other users participate in wellness applications and alters the mental state of a user by accounting for the context in which multiple users participate in a wellness application.

A method, non-transitory computer-readable storage medium, and computer system are disclosed for providing augmented wellness environment data to a client device. Location data is retrieved for a user and at least one other user is identified using the retrieved location data. The activity data for the users is retrieved and used to generate augmented wellness environment data for displaying an augmented wellness environment at the client device of the user. The augmented wellness environment can include a virtual rendered object. The augmented wellness environment data is provided to the client device for display.

Additional method, non-transitory computer-readable storage medium, and computer system are disclosed for generating an interactive wellness experience. An augmented wellness environment is generated at a media processing device, where the augmented wellness environment includes a rendered virtual object. Interactions between a user and at least one other user are accessed upon detecting that the user has completed a wellness experience. The augmented wellness environment is modified based on the accessed interactions.

In some embodiments, respective mood scores of the user and at least one other user can be determined based on biometric data of the respective users. An augmented wellness environment can be generated by outputting a first audio signal at a speaker coupled to a media processing device. The augmented wellness environment data may be updated based on the mood scores, where the updated augmented wellness environment data includes a second audio signal. The second audio signal can be provided to the client device (e.g., instead of the first audio signal). An augmented wellness environment can be additionally or alternatively generated by displaying the virtual object in a first color. The augmented wellness environment data can be updated based on the mood scores, where the updated augmented wellness environment data includes a second color. The second color can be provided to the client device for display (e.g., instead of the first color).

Interactions between the user and the at least one other user can include a joint wellness experience and characteristics of the joint wellness experience. The characteristics of the joint wellness experience that can be used to modify the augmented wellness environment include one or more of a location at which the joint wellness experience is performed, a duration of time during which the joint wellness experience is performed, a time of day at which the joint wellness experience begins, or biometric data of participants of the joint wellness experience. The location can be tracked by a global positioning system (GPS) sensor of a client device communicatively coupled to a media processing device at which the augmented wellness environment is generated. The biometric data can be monitored by one or more sensors coupled to media processing devices or client devices of the participants. Biometric data can include one or more of a heart rate or breathing rate.

A number of participants in the joint wellness experience can be determined in real time during the joint wellness experience. For example, a number by which the participant amount changes can be determined, where the number of participants of the joint wellness experience may change as users begin or end respective wellness experience within a predetermined distance of the participants of the joint wellness experience. In some embodiments, to modify the augmented wellness environment, an additional virtual object can be displayed upon determining that a number of participants of the joint wellness experience has increased or a presently displayed virtual object can be removed from display upon determining that the number of participants of the joint wellness experience has decreased.

A virtual object can be an avatar associated with the user. A number of wellness experiences that a user has completed can be determined and the avatar (e.g., the appearance of the avatar) can be modified based on the determined number of wellness experiences. The virtual object can be an AR object or a VR object. The expiration of a timer can be determined to detect that a user has completed a wellness experience, where the timer was initiated for a user-specified duration of time (e.g., a desired length of a guided meditation). A dynamic map can be generated, where the map includes icons corresponding to users within a predetermined distance of the user. The icons can also indicate whether the users are participating in a wellness experience.

### BRIEF DESCRIPTIONS OF THE DRAWINGS

The disclosed embodiments have other advantages and features which will be more readily apparent from the following detailed description of the invention and the appended claims, when taken in conjunction with the accompanying drawings, in which:

Figure (or “FIG.”) 1 illustrates an example embodiment of a media system.

FIG. 2 illustrates an example embodiment of a media processing device.

FIG. 3 illustrates an example embodiment of a media server.

FIG. 4 depicts an example embodiment of a wellness experience including breathwork.

FIG. 5 depicts an example embodiment of a wellness experience including a mindful walk.

FIG. 6 depicts an example embodiment of a user interface for managing rings of users.

FIG. 7 depicts an example embodiment of a user interface for locating users on a map.

FIG. 8 illustrates an example embodiment of a process for generating an interactive wellness experience.

FIG. 9 illustrates an example embodiment of a process for providing augmented wellness environment data to a client device for displaying an augmented wellness environment.

### DETAILED DESCRIPTION

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described.

A media system adaptively generates an augmented wellness experience based on the interactions between users. The media system modifies the wellness experience based on the interactions to alter (e.g., improve) a user's mood. As referred to herein, a “wellness experience” is a digitally augmented activity for changing an mental state, or “mood,” of a user (e.g., as measured by biometric data. Digital augmentation may include one or more of displaying graphical objects (e.g., virtual reality (VR) or augmented reality (AR) objects) or outputting audio (e.g., ambient music). Example activities that can be augmented are guided meditations, walks, breathwork, or focus games. The media system monitors for interactions between users while the users are engaged in wellness experiences. For example, the media system can monitor biometric activity of users participating in a digitally augmented activity together, which may be referred to as a “joint wellness experience.” During the joint wellness experience, the media system can determine moods or quantitative representations of the users' moods, which may be referred to herein as “mood scores,” and change the digital augmentation using the mood scores. The media system can also modify wellness experiences based on the users' locations, generating particular graphical objects or audio based on user locations (e.g., using conditional rules). In these ways, the media system integrates users' wellness with other users and their environment, eliminating isolation and welcoming community.

FIG. 1 is a block diagram of a media system 100 according to one embodiment. The media system 100 includes a network 120, a media server 130, one or more media processing devices 110 executing a virtual reality (VR) application 112 or an augmented reality (AR) application 114, and one or more client devices 140 executing a client application 142. In alternative configurations, different, additional, or fewer components may be in the media content system 100. For example, only one of the VR application 112 or the AR application 114 may be included at the media processing device 110.

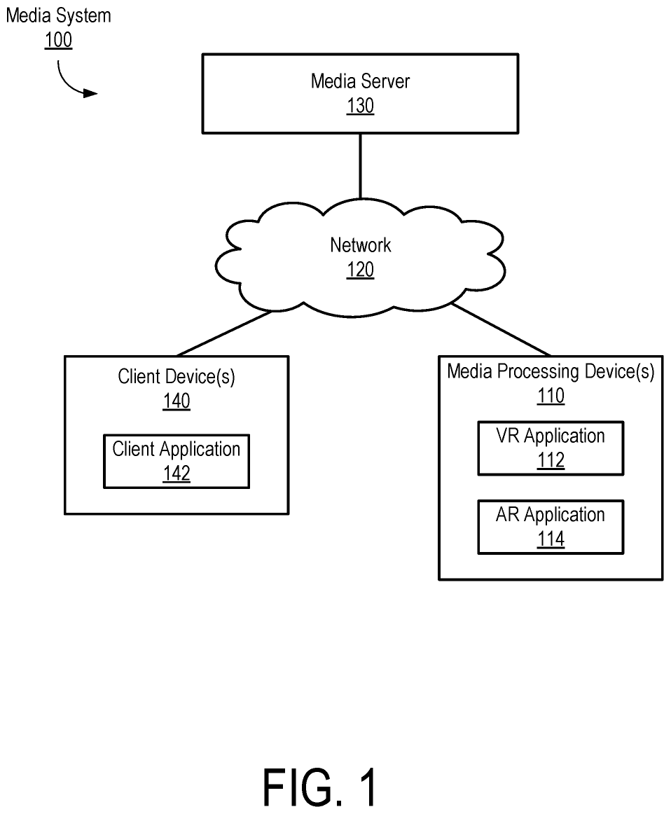

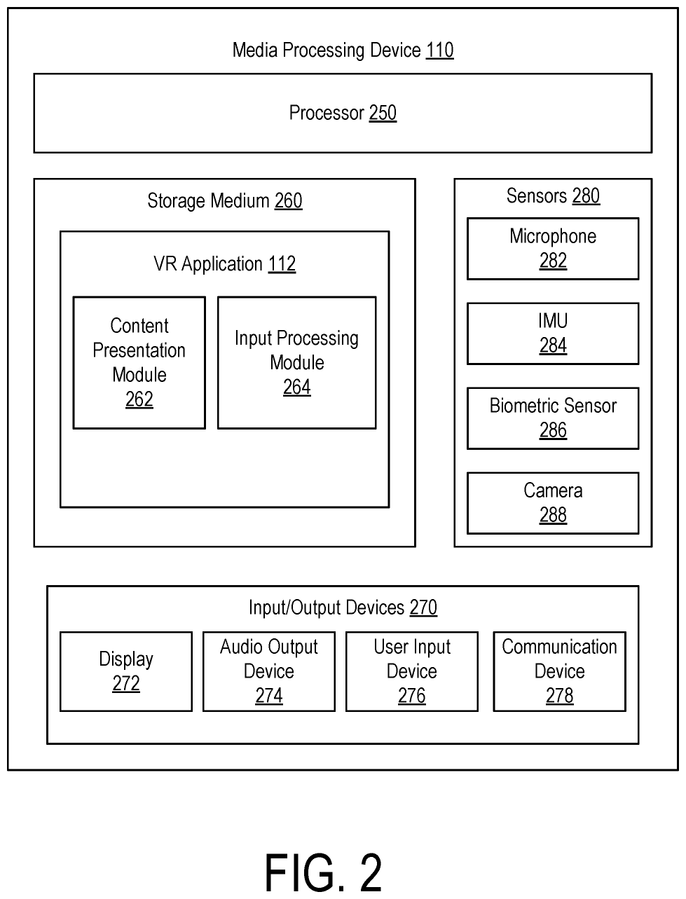

The media processing device 110 includes a computer device for processing and presenting media content such as audio, images, video, or a combination thereof. In an embodiment, the media processing device 110 is a head-mounted VR or AR device. The media processing device 110 may detect various inputs including voluntary user inputs (e.g., input via a controller, voice command, body movement, or other convention control mechanism) and various biometric inputs (e.g., breathing patterns, heart rate, etc.). The media processing device 110 may execute the VR application 112 or the AR application 114 that provides an immersive wellness experience to the user, which may include visual and audio media content. The VR application 112 or the AR application 114 may control presentation of media content in response to the various inputs detected by the media processing device 110. For example, the VR application 112 may adapt presentation of visual content as the user moves his or her head to provide an immersive wellness experience. An embodiment of a media processing device 110 is described in further detail below with respect to FIG. 2.

The client devices 140 are computing devices that execute a client application 142 providing a user interface to enable the user to input and view information that is directly or indirectly related to a wellness experience. For example, the client application 142 may enable a user to set up a user profile that becomes paired with the VR application 112 or the AR application 114. Furthermore, the client application 142 may present various surveys to the user before and after wellness experiences to gain information about the user's reaction to the wellness experience. Examples of a e client device 140 include a mobile device, tablet, laptop computer, desktop computer, gaming console, or other network-enabled computer device.

The media server 130 is one or more computing devices for delivering media content to the media processing devices 110 via the network 120 and for interacting with the client device 140. For example, the media server 130 may stream media content to the media processing devices 110 to enable the media processing devices 110 to present the media content in real-time or near real-time. Alternatively, the media server 130 may enable the media processing devices 110 to download media content to be stored on the media processing devices 110 and played back locally at a later time. The media server 130 may furthermore obtain user data about users using the media processing devices 110 and process the data to dynamically generate media content tailored to a particular user. Particularly, the media server 130 may generate media content (e.g., in the form of a wellness experience) that is predicted to improve a particular user's mood based on profile information associated with the user received from the client application 142 and a machine-learned model that predicts how users' moods improve in response to different wellness experiences.

The network 120 may include any combination of local area or wide area networks, using both wired or wireless communication systems. In one embodiment, the network 120 uses standard communications technologies or protocols. In some embodiments, all or some of the communication links of the network 120 may be encrypted using any suitable technique.

Various components of the media system 100 of FIG. 1 such as the media server 130, the media device 110, and the client device 140 can each include one or more processors and a non-transitory computer-readable storage medium storing instructions that, when executed, cause the one or more processors to carry out the functions attributed to the respective devices.

FIG. 2 is a block diagram illustrating an embodiment of a media processing device 110. In the illustrated embodiment, the media processing device 110 includes a processor 250, a storage medium 260, input/output devices 270, and sensors 280. Alternative embodiments may include additional or different components.

The input/output devices 270 include various input and output devices for receiving inputs to the media processing device 110 and providing outputs from the media processing device 110. In an embodiment, the input/output devices 270 may include a display 272, an audio output device 274, a user input device 276, and a communication device 278. The display 272 is an electronic device for presenting images or video content such as an LED display panel, an LCD display panel, or other type of display. The display 272 may be a head-mounted display that presents immersive VR content. The audio output device 274 may include one or more integrated speakers or a port for connecting one or more external speakers to play audio associated with the presented media content. The user input device 276 can be any device for receiving user inputs such as a touchscreen interface, a game controller, a keyboard, a mouse, a joystick, a voice command controller, a gesture recognition controller, or other input device. The communication device 278 includes an interface for receiving and transmitting wired or wireless communications with external devices (e.g., via the network 120 or via a direct connection). For example, the communication device 278 may have one or more wired ports such as a USB port, an HDMI port, an Ethernet port, etc. or one or more wireless ports for communicating according to a wireless protocol such as Bluetooth, Wireless USB, Near Field Communication (NFC), etc.

The sensors 280 capture various sensor data that can be provided as additional inputs to the media processing device 110. For example, the sensors 280 may include a microphone 282, an inertial measurement unit (IMU) 284, one or more biometric sensors 286, and a camera 288. The microphone 282 captures ambient audio by converting sound into an electrical signal that can be stored or processed by the media processing device 110. The IMU 284 is a device for sensing movement and orientation. For example, the IMU 284 may include a gyroscope for sensing orientation or angular velocity and an accelerometer for sensing acceleration. The IMU 284 may furthermore process data obtained by direct sensing to convert the measurements into other useful data, such as computing a velocity or position from acceleration data. In an embodiment, the IMU 284 may be integrated with the media processing device 110. Alternatively, the IMU 284 may be communicatively coupled to the media processing device 110 but physically separate from it so that the IMU 284 could be mounted in a desired position on the user's body (e.g., on the head or wrist).

The biometric sensors 286 are one or more sensors for detecting various biometric characteristics of a user, such as heart rate, breathing rate, blood pressure, temperature, or other biometric data. The biometric sensors may be integrated into the media processing device 110, separate sensor devices that may be worn at an appropriate location on the human body, or both. In one embodiment, the biometric sensors communicate sensed data to the media processing device 110 via a wired or wireless interface. The camera 288 may capture image or video data of the environment in which the media processing device 110 operates. The image or video data may be used by the media server 130 to render an augmented wellness experience. For example, an AR view of a user's real world environment, with AR objects overlayed onto the real world environment, may be generated using the AR application 114.

The storage medium 260 (e.g., a non-transitory computer-readable storage medium) stores a VR application 112 including instructions executable by the processor 250 for carrying out functions attributed to the media processing device 110 described herein. In an embodiment, the VR application 112 includes a content presentation module 262 and an input processing module 264. The content presentation module 262 presents media content via the display 272 and the audio output device 274. The input processing module 264 processes inputs received via the user input device 276 or from the sensors 280 and provides processed input data that may control the output of the content presentation module 262 or may be provided to the media processing server 130. For example, the input processing module 264 may filter or aggregate sensor data from the sensors 280 prior to providing the sensor data to the media server 130.

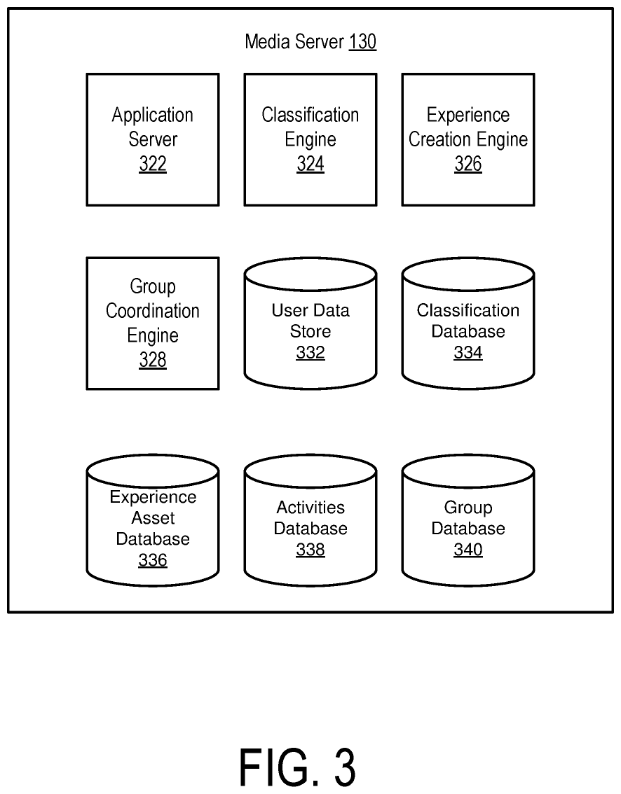

FIG. 3 illustrates an example embodiment of a media server 130. The media server 130 includes an application server 322, a classification engine 324, an experience creation engine 326, a user data store 332, a classification database 334, and an experience asset database 336. In alternative embodiments, the media server 130 may have different or additional components. Various components of the media server 130 may be implemented as a processor and a non-transitory computer-readable storage medium storing instructions that when executed by the processor causes the processor to carry out the functions described herein.

The experience asset database 336 stores digital assets that may be combined to create a wellness experience. Digital assets may include graphical objects, audio objects, and color palettes. Each digital asset may be associated with asset metadata describing characteristics of the digital asset and stored in association with the digital asset. For example, a graphical object may have attribute metadata specifying a shape of the object, a size of the object, or one or more colors associated with the object, etc. Graphical objects can be procedurally generated. For example, a graphical object may be a plant growing from a seed. Aspects of procedural generation may include morphology, texture, animation, links to other objects, reaction patterns to user input, etc.

Graphical objects may include a background scene or template (which may include still images or videos) and foreground objects (that may be still images, animated images, or videos). Foreground objects may move in three-dimensional space throughout the scene and may change in size, shape, color, or other attributes over time. Graphical objects may depict real objects or individuals, or may depict abstract creations.

Audio objects may include music, sound effects, spoken words, or other audio. Audio objects may include long audio clips (e.g., several minutes to hours) or very short audio segments (e.g., a few seconds or less). Audio objects may furthermore include multiple audio channels that create stereo effects.

Color palettes include a coordinated set of colors for coloring one or more graphical objects. A color palette may map a general color attributed to a graphical asset to specific RGB (or other color space) color values. By separating color palettes from color attributes associated with graphical objects, colors can be changed in a coordinated way during a wellness experience independently of the depicted objects. For example, a graphical object (or particular pixels thereof) may be associated with the color “green,” gut the specific shade of green is controlled by the color palette, such that the object may appear differently as the color palette changes.

Digital assets may furthermore have one or more scores associated with them representative of a predicted association of the digital asset with an improvement in mood that will be experienced by a user having a particular user profile when the digital asset is included in a wellness experience. In an embodiment, a digital asset may have a set of scores that are each associated with a different group of users (e.g., a “cohort”) that have similar profiles. Furthermore, the experience asset database 336 may track which digital assets were included in different wellness experiences and to which users (or their respective cohorts) the digital assets were presented.

In an embodiment, the experience asset database 336 may include user-defined digital assets that are provided by the user or obtained from profile data associated with the user. For example, the user-defined digital assets may include pictures of family members or pets, favorite places, favorite music, etc. The user-defined digital assets may be tagged in the experience asset database 336 as being user-defined and available only to the specific user that the asset is associated with. Other digital assets may be general digital assets that are available to a population of users and are not associated with any specific user.

The experience creation engine 326 generates the wellness experience by selecting digital assets from the experience asset database 336 and presenting the digital assets according to a particular time sequence, placement, and presentation attributes. For example, the experience creation engine 326 may choose a background scene or template that may be colored according to a particular color palette. Over time during the wellness experience, the experience creation engine 326 may cause one or more graphical objects to appear in the scene in accordance with selected attributes that control when the graphical objects appear, where the graphical objects are placed, the size of the graphical object, the shape of the graphical object, the color of the graphical object, how the graphical object moves throughout the scene, when the graphical object is removed from the scene, etc. Similarly, the experience creation engine 326 may select one or more audio objects to start or stop at various times during the wellness experience. For example, a background music or soundscape may be selected and may be overlaid with various sounds effects or spoken word clips. In some embodiments, the timing of audio objects may be selected to correspond with presentation of certain visual objects. For example, metadata associated with a particular graphical object may link the object to a particular sound effect that the experience creation engine 326 plays in coordination with presenting the visual object. Elements of the wellness experience may be generated procedurally. For example, wellness text in the form of guidance meditations can be generated using neural networks. Procedurally generated elements, such as affirmations, mantras, mindfulness meditations, etc., can be tailored to the user's profile. The experience creation engine 326 may furthermore control background graphical or audio objects to change during the course of a wellness experience, or may cause a color palette to shift at different times in a wellness experience. The collection of digital assets selected by the experience creation engine 326 for presentation may be referred to as “augmented wellness environment data.” The augmented wellness environment data may also include instructions to display digital assets at client devices.

The experience creation engine 326 may intelligently select which assets to present during a wellness experience, the timing of the presentation, and attributes associated with the presentation to tailor the wellness experience to a particular user. For example, the experience creation engine 326 may identify a cohort associated with the particular user, and select specific digital assets for inclusion in the wellness experience based on their scores for the cohort or other factors such as whether the asset is a generic asset or a user-defined asset. In an embodiment, the process for selecting the digital assets may include a randomization component. For example, the experience creation engine 326 may randomly select from digital assets that have at least a threshold score for the particular user's cohort. Alternatively, the experience creation engine 326 may perform a weighted random selection of digital assets where the likelihood of selecting a particular asset is weighted based on the score for the asset associated with the particular user's cohort, weighted based on whether or not the asset is user-defined (e.g., with a higher weight assigned to user-defined assets), weighted based on how recently the digital asset was presented (e.g., with higher weight to assets that have not recently been presented), or other factors. The timing and attributes associated with presentation of objects may be defined by metadata associated with the object, may be determined based on learned scores associated with different presentation attributes, may be randomized, or may be determined based on a combination of factors. By selecting digital assets based on their respective scores, the experience creation engine 326 may generate a wellness experience predicted to a have a high likelihood to improve the user's moods. Selection and presentation of objects can further be informed by accumulated tracked data about the user. For example, the experience creation engine 326 may track a user's energy levels throughout the day, week, month, or year, and drive content generation based on the tracked information.

In an embodiment, the experience creation engine 326 pre-renders the wellness experience before playback such that the digital objects for inclusion and their manner of presentation are pre-selected. Alternatively, the experience creation engine 326 may render the wellness experience in substantially real-time by selecting objects during the wellness experience for presentation at a future time point within the wellness experience. In this embodiment, the experience creation engine 326 may adapt the wellness experience in real-time based on biometric data obtained from the user in order to adapt the experience to the user's perceived change in mood. For example, the experience creation engine 326 may compute a mood score based on acquired biometric information during the wellness experience and may select digital assets for inclusion in the wellness experience based in part on the detected mood score.

The application server 322 obtains various data associated with users of the VR application 112 and the client application 142 during and in between wellness experiences and indexes the data to the user data store 332. For example, the application server 322 may obtain profile data from a user during an initial user registration process (e.g., performed via the client application 142) and store the user profile data to the user data store 332 in association with the user. The user profile information may include, for example, a date of birth, gender, age, and location of the user. Once registered, the user may pair the client application 142 with the VR application 112 so that usage associated with the user can be tracked and stored in the user data store 332 together with the user profile information.

The user data store 332 may also store avatars used to identify the user. The experience creation engine 326 may change the appearance of the avatar when particular conditions are met. The conditions may be related to time, location, people, activities, or any other suitable measure of a user's wellness experiences. For example, the avatar may change appearances each time the user completes a predetermined duration of wellness experiences in (e.g., changing appearances after 10 hours, 100 hours, and 1000 hours). In another example, the avatar may have a first appearance when the user is at a first location (e.g., home) and a second appearance when the user is at a second location (e.g., office). In some embodiments, the application server 322 may determine a number of wellness experiences that a user has completed and modify the user's avatar based on the determined number of wellness experiences.

In one embodiment, the tracked data includes survey data from the client application 112 obtained from the user between wellness experiences, biometric data from the user captured during (or within a short time window before or after) the user participating in a wellness experience, and usage data from the VR application 112 representing usage metrics associated with the user. For example, in one embodiment, the application server 322 obtains self-reported survey data from the client application 142 provided by the user before and after a particular wellness experience. The self-reported survey data may include a first self-reported mood score (e.g., a numerical score on a predefined scale) reported by the user before the wellness experience and a second self-reported mood score reported by the user after the wellness experience. The application server 322 may calculate a delta between the second self-reported mood score and the first self-reported mood score, and store the delta to the user data store 332 as a mood improvement score associated with the user and the particular wellness experience. Additionally, the application server 322 may obtain self-reported mood tracker data reported by the user via the client application 142 at periodic intervals in between wellness experiences. For example, the mood tracker data may be provided in response to a prompt for the user to enter a mood score or in response to a prompt for the user to select one or more moods from a list of predefined defined moods representing how the user is presently feeling. The application server 322 may furthermore obtain other text-based feedback from a user and perform a semantic analysis of the text-based feedback to predict one or more moods associated with the feedback.

The application server 322 may furthermore obtain biometric data from the media processing device 110 that is sensed during a particular wellness experience. Additionally, the application server 322 may obtain usage data from the media processing device 110 associated with the user's overall usage (e.g., characteristics of wellness experiences experienced by the user, a frequency of usage, time of usage, number of experiences viewed, etc.). All of the data associated with the user may be stored to the user data store 332 and may be indexed to a particular user and to a particular wellness experience.

The classification engine 324 classifies data stored in the user data store 332 to generate aggregate data for a population of users. For example, the classification engine 324 may cluster users into user cohorts. A user cohort is a group of users having sufficiently similar user data in the user data store 332 (e.g., having user data for which a similarity metric is less than or greater than a threshold). When a user first registers with the media experience server 130, the classification engine 324 may initially classify the user into a particular cohort based on the user's provided profile information (e.g., age, gender, location, etc.). As the user participates in wellness experiences, the user's survey data, biometric data, and usage data may furthermore be used to group users into cohorts. Thus, the users in a particular cohort may change over time as the data associated with different users is updated. Likewise, the cohort associated with a particular user may shift over time as the user's data is updated.

The classification engine 324 may furthermore aggregate data associated with a particular cohort to determine general trends in survey data, biometric data, or usage data for users within a particular cohort. Furthermore, the classification engine 324 may furthermore aggregate data indicating which digital assets were included in wellness experiences experienced by users in a cohort. The classification engine 324 may index the aggregate data to the classification database 334. For example, the classification database 334 may index the aggregate data by gender, age, location, experience sequence, and assets. The aggregate data in the classification database 334 may indicate, for example, how mood scores changed before and after wellness experiences including a particular digital asset. Furthermore, the aggregate data in the classification database 334 may indicate, for example, how certain patterns in biometric data correspond to surveyed results indicative of mood improvement.

The classification engine 324 may furthermore learn correlations between particular digital assets included in experiences viewed by users within a cohort and data indicative of mood improvement. The classification engine 324 may update the scores associated with the digital assets for a particular cohort based on the learned correlations.

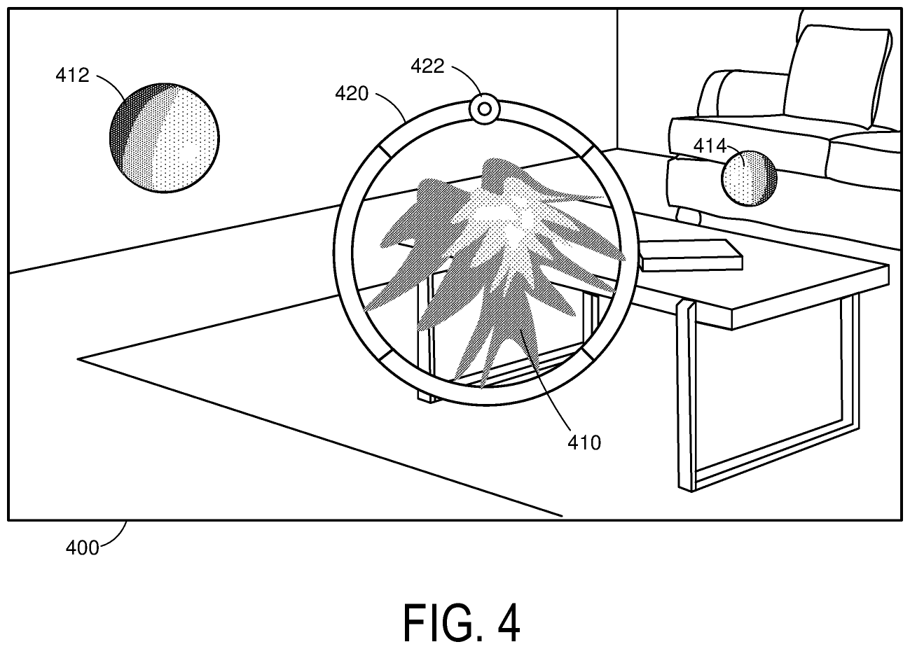

The activities database 338 stores parameters for generating wellness experiences. Wellness experiences may include an activity to control breath, an activity to control focus, meditating, walking, or a combination thereof. For an activity to control breath, which may also be referred to as “breathwork,” the activities database 338 may store particular instructions (e.g., timing sequences) for breathing types (e.g., 4-7-8 breathing, box breathing, humming breath, belly breath, etc.), where the instructions can include text or graphics for display or prerecorded audio instructions. An example of a breathwork activity is depicted in FIG. 4. For an activity to control focus, which may also be referred to as a “focus game,” the activities database 338 may store a mapping of experience assets to a particular focus activity to create a game experience or daily reflection. For a meditating activity, the activities database 338 may store visual guides, meditation topics, information on various meditation teachers, meditation instructions from the meditation teachers, ambient music, sound frequencies, or any other suitable parameter for creating an augmented wellness experience during a user's meditation. For a walking activity, which may also be referred to herein as a “mindful walk,” the activities database 338 may store information on various meditation teachers, meditation instructions from the meditation teachers, ambient music, sound frequencies, pre-determined paths for walking, mapping between experience assets and locations on particular paths, or any other suitable parameter for creating an augmented wellness experience while a user walks.

The activities database 338 may store locations associated with one or more users' wellness experiences. The locations may be user-specified or pre-determined for use by a user during their wellness experience. The locations may be associated with global positioning system (GPS) coordinates such that a user may access a particular activity at the specific locations. The locations may have a particular type, where each type is associated with a set of parameters for a wellness experience.

A first type of location stored in the activities database 338 may be a “sacred space,” which may be a location associated with experience assets generated as users participate in wellness experiences at the location. The sacred space may be an individual's personal space, where the generated experience assets are generated by the individual. Alternatively, the sacred space may be a group's shared space, where the generated experience assets are generated by two or more users who have completed wellness experiences at the location. The activities database 338 may store a mapping of generated experience assets and a location of type “sacred space” for access by the experience creation engine 326 to generate experience assets in a user's wellness experience performed at sacred space locations.

A second type of location stored in the activities database 338 may be a “serenity garden,” which may be a location associated with a specific experience asset, a “seed,” that is generated as users participate in wellness experiences at the location. Similar to a sacred space, the serenity garden may be a user's individual space or a group's shared space. The activities database 338 may store a mapping of generated seeds, which may become a different experience asset (e.g., a virtual flower) over time, and a location of type “serenity garden” for access by the experience creation engine 326 to generate one or more of seeds and flowers, or any suitable type of graphical object associated with gardens, in a user's wellness experienced performed at serenity garden locations.

In some embodiments, the growth of a seed is impacted by the wellness activities of one or more users in the serenity garden. For example, the rate of growth of a plant from the seed may be dependent on the amount of time the user spends on wellness activities in the serenity garden (e.g., the amount of growth may be proportional to the number wellness activities or the amount of time spent performing wellness activities in the serenity garden). Conversely, if the users do not perform any wellness activities in the wellness garden for an extended period of time (e.g., more than three days, a week, a month, etc.), the plant way begin to wither and die or reduce in size. As another example, visual aspects such as the color, shape, and type of the virtual plants, fruits, seeds, or the like growing in the serenity garden, may be modified by a user's mood (whether self-reported or determined from biometric data) while in the serenity garden.

A third type of location stored in the activities database 338 may be a “cosmic portal,” which may be a location associated with a multiplier that increases the amount of reward a user is given for completing a wellness experience. A wellness experience may be associated with a quantitative value that summarizes quantitative or qualitative aspects of the user's wellness experience (e.g., how long the experience lasted, the accuracy of the user's breath when following breathing instructions, etc.). The quantitative value may be a score or a number of points that the user may accumulate over time, as the user performs wellness experiences. The increased reward may be an increased quantitative value received for performing a wellness experience at the cosmic portal. The activities database 338 may store a list of GPS coordinates associated with cosmic portals. The experience creation engine 326 may determine that a user is located at GPS coordinates of a cosmic portal and access a multiplier stored at the activities database 338 to determine an amount by which the quantitative value of a user's wellness experience performed at the cosmic portal is increased. The activities database 338 may store different multipliers for different cosmic portals. In one example, the multiplier associated with one or more cosmic portals is a multiplier by 3 (e.g., a user receiving 10 points at a location that is not a cosmic portal may receive 30 points at a cosmic portal). Additional parameters associated with the cosmic portal may be stored in the activities database 338. For example, the cosmic portal may increase the reward received by a user for a specific time limit, where the time limit parameter is stored in the activities database 338.

The group coordination engine 328 manages a wellness experience with two or more participants, which may be referred to as a “joint wellness experience.” The group coordination engine 328 may determine that users are available to participate in a joint wellness experience by receiving location data (e.g., GPS data) indicating that the users are within a predetermined distance of one another (e.g., within a quarter mile radius of one of the users) and determining if the users are or can be available to perform a joint wellness experience. Alternatively, users may be assigned to a group if they are within a predetermined geographic area (e.g., the same city block or building). The group coordination engine 328 may receive location data from the client devices of users over the network 120. Using the received location data, the group coordination engine 328 may determine a set of users within a predetermined distance of one another. For example, the group coordination engine 328 determines two users that are within a predetermined distance of one another, and determines any additional users that are within the same predetermined distance of the two users. The group coordination engine 328 may determine which of the group of users within proximity of each other (e.g., combining the two users and the additional users) are currently or can be available to perform a joint wellness experience. For example, the group coordination engine 328 may determine, based on a user's preferences (e.g., stored in the user data store 332) not to perform joint wellness experiences, that a user is unavailable for a joint wellness experience.

The group coordination engine 328 may generate notifications at client devices, where the notifications include invitations to join a joint wellness experience. The group coordination engine 328 may determine at which client devices to generate the notification. The group coordination engine 328 may use a statistical model or machine learning model created using historical user responses to invitations to join joint wellness experiences. For example, the group coordination engine 328 may log the user responses to invitations (e.g., accept or decline) and context of the invitation (e.g., information about the user, location at which the invitation was sent, time of day at which the invitation was sent, on what type of device (e.g., smartphone, tablet, etc.) the invitation was sent, etc.). Using the logged information, the group coordination engine 328 may create a statistical model or machine learning model for determining a likely response based on context available to the group coordination engine 328. For example, the group coordination engine 328 may input context such as a time of day and a user identifier into a model to determine that the user associated with the user identifier is unlikely to accept the invitation. In turn, the group coordination engine 328 may determine not to generate the notification at the user's client device, thus not inviting the user to a joint wellness experience. Alternatively, the group coordination engine 328 may determine that a user is indeed likely to accept the invitation and in response, generate the notification at the user's client device to invite the user to a joint wellness experience. This determination whether or not to send the invitation may save communication bandwidth between the media server 130 and the client devices 140 in addition to processing resources and memory for handling the notifications at both the media server 130 and the client devices 140.

The group coordination engine 328 may add users to a joint wellness experience that has already been initiated by the group coordination engine 328 or create a new joint wellness experience. The group coordination engine 328 may determine a type of activity for the joint wellness experience before or after determining users that are within proximity of each other to perform the joint wellness experience. The group coordination engine 328 may determine a type of activity to recommend for the new joint wellness experience. In some embodiments, the group coordination engine 328 may determine an activity from one or more of breathwork, meditation, mindful walking, or focus games. The group coordination engine 328 may determine an activity that a group of users are most likely to perform together. In some embodiments, the group coordination engine 328 may use a weighted decision of activities that individual users of the group are likely to perform to determine the most likely activity that the group will perform. The group coordination engine 328 may use a model (e.g., statistical, machine learning, or any suitable predictive model) for determining a likelihood of one or more users performing an activity. The model may be trained on historical data of the users' activities performed and the contexts in which they were performed (e.g., time of day, location, whether a notification generated by the media server 130 caused the user to perform the activity, a number of users the activity was performed with, etc.).

The group coordination engine 328 may initiate a joint wellness experience using an activity determined by the group coordination engine 328 or using an activity specified by one of the users in a group of users. The group coordination engine 328 may access parameters of the activity from the activities database 338. In one example of initiating a joint wellness experience, the group coordination engine 328 determines to initiate a mindful walk for a group of users and accesses parameters of the mindful walk from the activities database 338. The group coordination engine 328 can provide for display a map of the path to be walked, as accessed from the activities database 338, to the client devices 140 of the users in the group. The group coordination engine 328 may begin to output ambient music associated with the mindful walk at the client devices 140 by providing an audio file to the client devices 140 for output at the speakers of the devices 140. In another example of initiating a joint wellness experience, the group coordination engine 338 determines to initiate a breathwork activity for a group of users and accesses parameters of the breathwork activity from the activities database 338. The group coordination engine 328 can provide for display graphic instructions at the client devices 140 of the users, instructing the users to breath in synchrony according to a particular breath type (e.g., 4-7-8 breaths).

The group coordination engine 328 may coordinate with the experience creation engine 526 to initiate and manage a joint wellness experience. For example, the group coordination engine 328 may provide instructions to the experience creation engine 326, upon the start of and throughout a joint wellness experience, to start providing audio and visuals to users' client devices 140. The group coordination engine 328 may cause the experience creation engine 326 to output the same audio and visual components at each of the client devices 140 of users in the joint wellness experience. In this way, users participating in a joint wellness experience can have a shared experience. The group coordination engine 328 may change the instructions provided to the experience creation engine 326 based on information received from the client devices 140 related to the user's performance of the joint wellness experience. A user's performance in a joint wellness experience may include joining or leaving a joint wellness experience, measured biometrics, the difference between the measured biometrics and a target biometric (e.g., empirical vs instructed breathing rate), or any suitable attribute describing a user's experience in a joint wellness experience. For example, the group coordination engine 328 receives information from a client device that a user has stopped performing the activity at their device, leaving the joint wellness experience, and in response, the group coordination engine 328 instructs the experience creation engine 326 to reduce a number of experience assets displayed to the remaining users participating in the joint wellness experience.

The group coordination engine 328 can determine a combined mood score based on the respective individual mood scores of participants in a joint wellness experience or based directly on the biometric activity or any other suitable measured activity of the participants of the joint wellness experience. The combined mood score may be an interaction between users of a joint wellness experience that causes the group coordination engine 328 to modify a joint wellness experience for the participants. In some embodiments, the group coordination engine 328 may use a model to determine the combined mood score based on biometric activity or other measured user activity (e.g., the users' paces during a mindful walk). The measured user activity accessed by the group coordination engine 328 may be referred to as “activity data.” The activity data may be stored in the user data store 332. The model may be a rules-based model, a statistical model, a machine learning model, or any suitable model for correlating measured user activity to a combined mood score. In one example, the group coordination engine 328 trains a machine learning model using historical measured user activity (e.g., sets of users' biometric heart rates and breathing rates) labeled with a combined mood score label. The trained machine learning model may be applied to currently measured user activity to output a combined mood score. The trained machine learning model may be retrained based on user feedback (e.g., survey data) of the user's moods or the satisfaction with the wellness experience. For example, an indication of dissatisfaction may be used to retrain the machine learning model to decrease the likelihood that the measured user activity is associated with the combined mood score that contributed to the unsatisfactory wellness experience. Similarly, an indication of satisfaction may be used to retrain the machine learning model to increase the likelihood that the measured user activity is associated with the determined combined mood score that contributed to the satisfactory wellness experience.

The group coordination engine 328 may determine, in substantially real time during a joint wellness experience, a number of participants in a joint wellness experience. The group coordination engine 328 may determine that the number of participants has changed as users begin and end respective wellness experiences on their client devices, which may correspond to entering and leaving a joint wellness experience managed by the group coordination engine 328. In some embodiments, the group coordination engine 328 determines that a user has entered a joint wellness experience by determining that the user is within a predetermined distance of existing participants of the joint wellness experience and that the user is currently engaged in their individual wellness experience. In this manner, a joint wellness experience does not necessarily cause the group coordination engine 328 to instruct the experience creation engine 326 to render the same wellness experience environment (e.g., the same experience assets, the same music, etc.) across all devices of users participating in a joint wellness experience. Rather, in some embodiments, the joint wellness experience may be a collection of users within a physical proximity of one another engaged in individual wellness experience. In some embodiments, the application server 322 may determine the number of participants in a joint wellness experience in substantially real time during the joint wellness experience.

In some embodiments, the group coordination engine 328 may facilitate a joint wellness session between users who are not within a predetermined physical distance of one another. For example, the group coordination engine 328 may receive a request to initiate a joint wellness session from a first user located at a first location with a second user located at a second location, the first and second locations greater than the predetermined distance from one another. In response to receive the request, the group coordination engine 328 may generate a notification at the client device of the second user, where the notification includes buttons to accept or deny the first user's request to begin a joint wellness session. The group coordination engine 328, in response to receiving the second user's indication that they accept the first user's request, may initiate the joint wellness session between the two users by rendering the same wellness experience environment (e.g., the same experience assets and audio) for the two users at their respective media processing devices.

The group coordination engine 328 may manage user-created networks of users, which may be referred to as “rings.” A ring of users may include two or more users. The group coordination engine 328 may maintain a data structure associating users to respective rings. Users may specify a name of a ring to the group coordination engine 328, which may be stored in the data structure. For example, a user may create a ring of “Family” and add other users into the ring. The group coordination engine 328 receives the user's request to create a ring, which includes the name “Family” and user identifiers for one or more users that the user has requested to include in the “Family” ring. The data structures of rings may be stored in the group database 340. The group coordination engine 328 may recommend users to add to a new or existing ring. The group coordination engine 328 can determine recommended users using the classification cohorts determined by the classification engine 324. The group coordination engine 328 may use users participating in a joint wellness experience to recommend users for a ring. For example, after the group coordination engine 328 terminates a joint wellness experience, the group coordination engine 328 may send a notification to the participants' client devices inviting each of the participants into a new or existing ring. The group coordination engine 328 may use data about the joint wellness experiences to determine to recommend users form a ring. For example, the group coordination engine 328 may use a threshold frequency or threshold number of occurrences for a joint wellness experience participated in by two or more of the same users to suggest that the users form a ring.

FIG. 4 depicts an example embodiment of a wellness experience including breathwork. An augmented reality view 400 can be generated at a media processing device (e.g., the media processing device 110) using a media server (e.g., the media server 130). The media server 130 may generate experience assets 410, 412, and 414 for display at the media processing device 110. In one example, the media processing device 110 is communicatively coupled to a client device (e.g., the client device 140), and the experience creation engine 326 accesses the experience assets 410-414 from the experience asset database 336 to provide to the client device 140, which further provides the assets 410-414 for display at the media processing device 110. In the depicted example, the media server 130 is providing the wellness experience to the media processing device 110 within the user's home, and the experience assets 410-414 (e.g., augmented reality objects) are generated overlaying the user's furniture (e.g., coffee table, sofa, etc.).

The wellness experience shown in the AR view 400 is an example breathwork activity, where the instructions for performing the activity are provided through instruction graphics 420 and 422. The instruction graphic 420 is a circular progress bar in which the instruction graphic 422 travels. The instruction graphic 420 is partitioned into different segments, which may correspond to different stages of a breath cycle, where one complete breath cycle is represented by the entire circle. The instruction graphic 422 travels along the different segments to instruct the user to breath a particular way at each segment (e.g., a first stage of inhaling, a second stage of inhaling, a stage of holding the breath, and a stage for exhaling).

As the user engages with the wellness experience, breathing according to the instructions, the media processing device 110, the client device 140, or any other biometric sensor coupled to one or more of the media processing device 110, the client device 140, or the media server 130 may measure biometric activity of the user (e.g., their breathing rate, heart rate, temperature, oxygen level, perspiration level, blood pressure, etc.). The media server 130 may receive the measured biometric activity of the user and modify the wellness experience based on the measured biometric activity (e.g., to cause a change in the user's mood). For example, the experience creation engine 326 may generate the experience asset 410 in a first color or first combination of colors or generate the experience asset 410 in a first movement pattern.

After receiving biometric activity and determining, from the received biometric activity, that the user's mood is worsening (e.g., heart rate or breathing rate increasing, indicating that the user has become more agitated, anxious, or other mental state that is not suitable for a state of wellness), the experience creation engine 326 may modify the first color or first combination of colors into a second color or second combination of colors that is associated with changing the user's mood. Additionally or alternatively, the experience creation engine 326 may modify the first movement pattern of the experience asset 410 into a second movement pattern (e.g., a slowly rhythm of movement) to induce a change in the user's mood. The modification may be based on previous modifications that have resulted in a desired change in the user's mood (e.g., previous modifications that have been followed by a decreasing heart rate or breathing rate).

In some embodiments, the experience creation engine 326 may reinforce good performance of the user that follows an ideal performance by generating additional experience assets or deter poor performance of the user that does not follow the ideal performance by removing existing experience assets that have been generated. For example, the experience creation engine 326 determines, from the received biometric activity, the user is following the breathing instructions as indicated by the graphics 420 and 422, the experience creation engine 326 may generate additional experience assets such as the assets 412 and 414 (e.g., objects with the appearance of planets) or increase the size of an existing asset (e.g., the asset 410). In another example, the experience creation engine 326 determines, from the received biometric activity, that the user's breath has strayed from the breathing instructions and removes one of the assets 412 or 414 or decreases the size of an existing asset (e.g., the size of the asset 410).

The breathwork activity may be participated in by a user as an individual wellness experience or by two or more users as a joint wellness experience. In an example of a joint wellness experience, two users are seated in the living room depicted in the background of the AR view 400. One of the users is seated at the angle as shown in FIG. 4, and another user may be seated from a different angle not depicted in FIG. 4. The group coordination engine 328 may manage the joint wellness activity, instructing the experience creation engine 326 to generate the same wellness experience environment for the two users, where the environment includes the experience assets 410-414 and the instruction graphics 420 and 422. The experience creation engine 326 may receive the biometric activity for each user and determine a combined mood score for the users participating in the joint wellness activity.

In one example, a first user's biometric activity indicates that their breathing rate and heart rate is within a predetermined range associated with an ideal, calm mental state and the second user's biometric activity indicates that their breathing rate and heart rate is outside of the predetermined range and instead, is in a different predetermined range associated with a non-ideal, stressed mental state. The experience creation engine 326 may determine to modify the wellness experience environment. The experience creation engine 326 may modify one or more of the experience assets 410-414 based on the combined mood score for the two users. For example, the experience creation engine 326 removes one of the displayed experience assets because the second user's mood score has increased a difference between the combined mood score and a target mood score and will redisplay the removed experience asset once the combined mood score is closer to the target mood score (e.g., less than a threshold difference). In another example, the experience creation engine 326 changes the color of an asset based on the combined mood score, where the modified color is determined by the experience creation engine 326 to decrease the difference between the combined mood score and the target mood score.

The experience creation engine 326 may also modify an audio signal output during the wellness experience to cause the difference between the combined mood score and the target mood score to decrease. For example, the experience creation engine 326 may determine that the second user has previously lowered their heart rate and breathing rate in response to music at lower frequencies, and in response, the experience creation engine 326 may select an audio track with a lower frequency of sounds (e.g., changing the current ambient soundtrack from the sound of flutes to a different soundtrack with the sound of gongs).

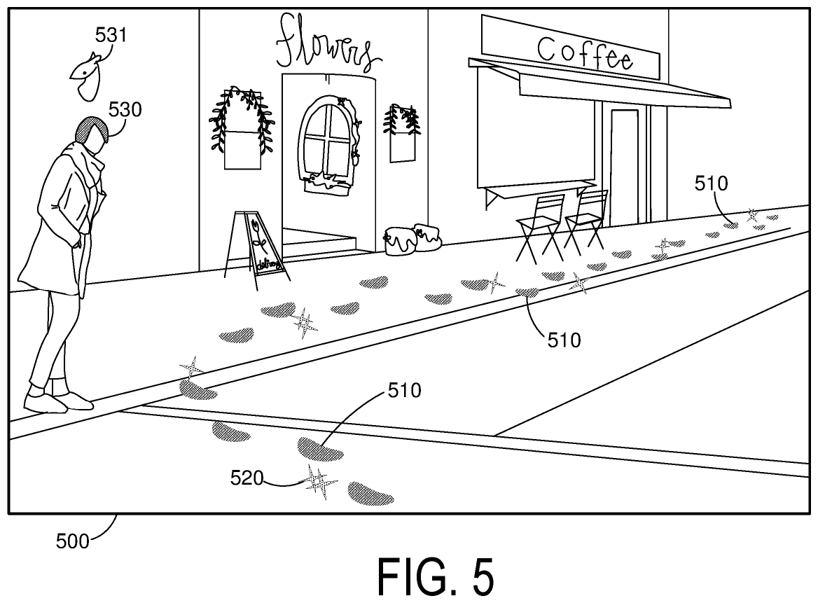

FIG. 5 depicts an example embodiment of a wellness experience including a mindful walk. An augmented reality view 500 can be generated at a media processing device (e.g., the media processing device 110) using a media server (e.g., the media server 130). The media server 130 may generate an instruction graphic 510 for display at the media processing device 110. The instruction graphic 510 may be an augmented reality object indicating a path that the user follows on a walk. In one example, the media processing device 110 is communicatively coupled to a client device (e.g., the client device 140), and the experience creation engine 326 accesses the instruction graphic 510 from the experience asset database 336 to provide to the client device 140, which further provides the instruction graphic 510 for display at the media processing device 110. In the depicted example, the media server 130 is providing the wellness experience to the media processing device 110 while the user is walking around a town (e.g., with a flower shop and a coffee shop), and the instruction graphic 510 is generated overlaying the real world objects (e.g., the crosswalk and the sidewalk). While an augmented reality view is depicted in FIG. 5, the media server 130 may also generate a virtual reality view for a mindful walk. For example, the user may be on a fitness equipment (e.g., a treadmill) that is communicatively coupled to one or more of the media processing device 110, the client device 140, or the media server 130. The speed at which the user is walking may be provided from the treadmill to the media server 130, which can modify the wellness experience using biometric activity and the movement activity as provided by the treadmill.

As the user engages with the wellness experience, walking according to the instruction graphic 510, the media processing device 110, the client device 140, or any other biometric sensor coupled to one or more of the media processing device 110, the client device 140, or the media server 130 may measure biometric activity of the user. The media server 130 may receive the measured biometric activity of the user and modify the wellness experience based on the measured biometric activity (e.g., to cause a change in the user's mood). For example, the experience creation engine 326 may generate the instruction graphic 510 in a first color or first combination of colors or generate an experience asset 520 (e.g., sparkles around footprints instructing the user to walk in a certain path).

After receiving biometric activity and determining, from the received biometric activity, that the user's mood is worsening (e.g., heart rate or breathing rate increasing, indicating that the user has become more agitated, anxious, or other mental state that is not suitable for a state of wellness), the experience creation engine 326 may modify the first color or first combination of colors into a second color or second combination of colors that is associated with changing the user's mood. Additionally or alternatively, the experience creation engine 326 may modify the number of experience assets (e.g., the number of sparkles) to induce a change in the user's mood. The modification may be based on previous modifications that have resulted in a desired change in the user's mood (e.g., previous modifications that have been followed by a decreasing heart rate or breathing rate).

In some embodiments, the experience creation engine 326 may reinforce good performance of the user that follows an ideal performance by generating additional experience assets or deter poor performance of the user that does not follow the ideal performance by removing existing experience assets that have been generated (e.g., adding or removing sparkles). For example, the experience creation engine 326 determines, from the received biometric activity, movement activity (e.g., via IMU sensors), or GPS data, that the user is following the walking instructions as indicated by the instruction graphic 510. In some embodiments, the experience creation engine 326 modifies the instruction graphic 510 based on sensor data indicating the user's walking pace. For example, the experience creation engine 326 colors the graphic 510 in at least three different colors at corresponding segments, where the segments can change as the user walks: a first segment of a path that the user should already be traveling on according to an ideal pace, a second segment of a path that the user has already walked on, and a third segment of the path that the user will eventually travel on according to the ideal pace.

The experience creation engine 326 may generate additional experience assets such as the asset 520 as the user maintains a pace according to the ideal pace of the instruction graphic 510. For example, generating more sparkles as the user maintains the pace and reducing the sparkles as the user strays from the pace. The experience creation engine 326 may modify audio provided during the mindful walk based on the pace traveled. For example, the experience creation engine 326 may decrease the volume of the audio as the user strays from the instructed pace and keep the volume of the audio at a desired level (e.g., as set by the user) as the user maintains the instructed pace.

The mindful walk may be participated in by a user as an individual wellness experience or by two or more users as a joint wellness experience. In an example of a joint wellness experience, two users are walking together along the path indicated by the instruction graphic 510. The two users may be next to one another or separated from one another (e.g., one is 70% done with the mindful walk's path and another is 10% done with the path). The group coordination engine 328 may manage the joint wellness activity, instructing the experience creation engine 326 to generate the same wellness experience environment for the two users, where the environment includes the instruction graphic 510, the experience asset 520, and an audio signal.

The experience creation engine 326 may receive the biometric activity or movement activity for each user and determine a combined mood score for the users participating in the joint wellness activity. In one example, a first user's biometric and movement activity indicates that their breathing rate and walking pace is within a predetermined range associated with an ideal, calm mental state and the second user's biometric activity indicates that their breathing rate and walking pace is outside of the predetermined range and instead, is in a different predetermined range associated with a non-ideal, agitated mental state. In response, the experience creation engine 326 may determine to modify the wellness experience environment. The experience creation engine 326 may modify one or more of the instruction graphic 510 or the experience asset 520 based on the combined mood score for the two users. The experience creation engine 326 may also modify an audio signal output during the wellness experience to cause the difference between the combined mood score and the target mood score to decrease.

The group coordination engine 328 may determine users nearby the user as the user walks along the path indicated by the instruction graphic 510. For example, the group coordination engine 328 may receive GPS coordinates from a client device of a user 530 who is not participating in the mindful walk depicted in the AR view 500. The group coordination engine 328 may determine, based on GPS coordinates from the user participating in the mindful walk (e.g., whose view is the view 500) and the GPS coordinates of the user 530, that the two users are within a predetermined distance of one another. In response, the group coordination engine 328 may instruct the experience creation engine 326 to generate an avatar 531 of the user 530 near the user 530, indicating that the user 530 is an individual who participates in wellness experiences through the media server 130. The group coordination engine 328 may determine whether to invite the user 530 to the mindful walk. For example, the group coordination engine 328 may, using information communicating to it through the operating system of the client device 140, determine that the user 530 is on a phone call and in response, the group coordination engine 328 may determine not to invite the user 530 to the mindful walk. If the group coordination engine 328 invites the user 530 to the walk and the user 530 accepts, the group coordination engine 328 may instruct the experience creation engine 326 to generate a wellness experience environment similar to the view 500 (e.g., having the instruction graphic 510 for the same path and the experience asset 520).

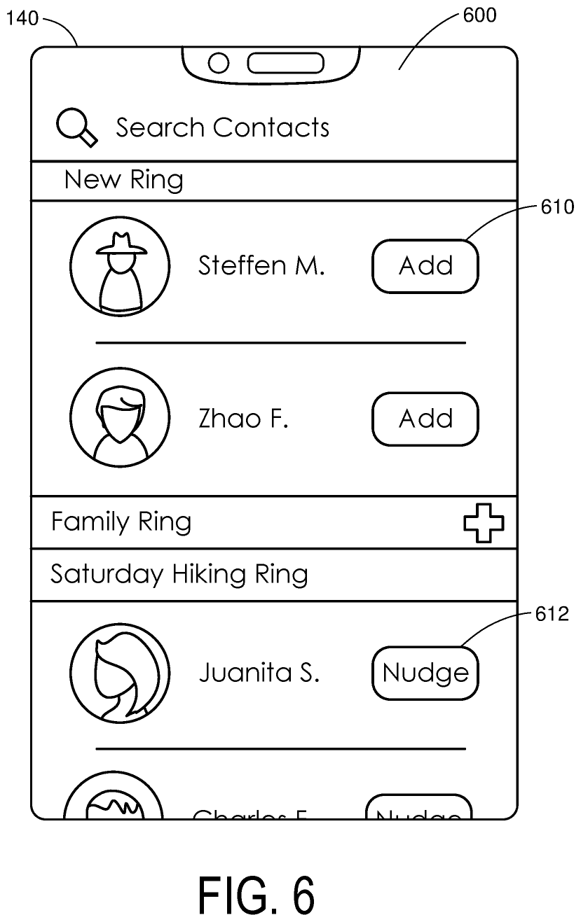

FIG. 6 depicts an example embodiment of a user interface for managing rings of users. The user interface 600 is a graphical user interface (GUI) generated at a client device 140 communicatively coupled to the media server 130. The group coordination engine 328 of the media server 130 can provide the GUI for display at the client device 140. The interface 600 includes a list of users that can be added to a ring by a user selection of a GUI element 610 (e.g., an “add” button). The interface 600 includes a list of users that are already in existing rings (e.g., family ring and Saturday hiking ring). Within the list of users in existing rings, the interface 600 includes a GUI element 612 (e.g., a “nudge” button) that a user may select to cause the group coordination engine 328 to generate a notification at the client device of a selected user, where the notification reminds the selected user to engage in a wellness experience. For example, a user selects the GUI element 612 to remind Juanita to be more mindful.

The group coordination engine 328 receives an indication that the user has selected the GUI element 612, where the indication includes the user identifiers for the user who selected the GUI element 612 and Juanita's user identifier. The group coordination engine 328 may then generate a notification at Juanita's client device, identifying the device based on Juanita's user identifier. The notification may include a message of which user has generated the notification (e.g., using the user's identifier) and a recommended wellness experience in which Juanita should engage. In some embodiments, the group coordination engine 328 may confirm that Juanita has engaged in a wellness experience in response to the generated notification and provide a reward to the user who reminded Juanita (e.g., providing points to the user, which in turn increase or modify the experience assets that the user has access to).

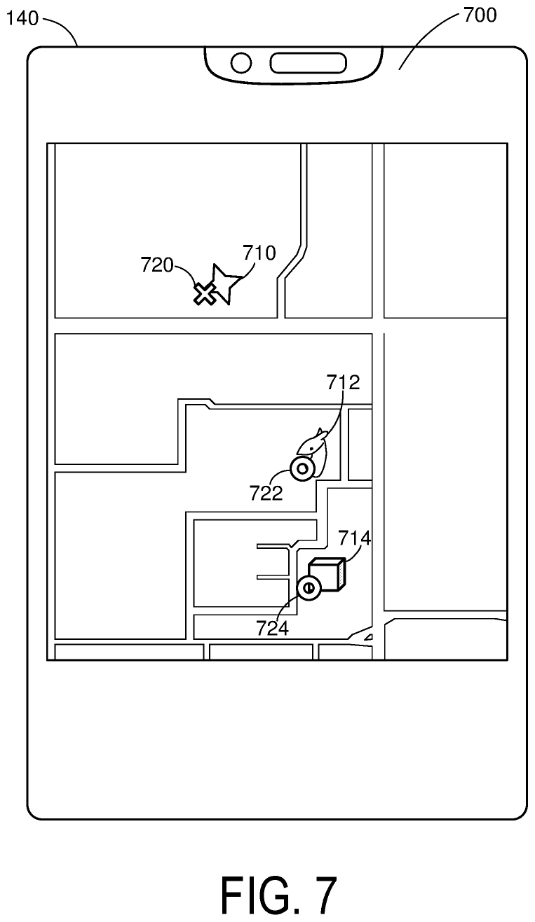

FIG. 7 depicts an example embodiment of a user interface for locating users on a map. The media server 130 may provide for display the user interface 700, which may be a GUI, on the client device 140. The group coordination engine 328 may generate an interactive map (e.g., a combination of physical and road maps). The map may include icons 710-714 at locations on the interactive map where users are located. In some embodiments, the icons may be user-selected. For example, the icons may be user avatars that may be stored in the user data store 332 and associated with corresponding experience assets stored in the experience asset database 336. When the experience creation engine 326 is rendering an environment for a wellness experience (e.g., joint or individual), the experience creation engine 326 may also use the user avatars in the wellness experience (e.g., as shown in FIG. 5 through the avatar 531).

The group coordination engine 328 may also render availability icons next to respective user avatars. For example, next to the icons 710-714, the group coordination engine 328 renders availability icons 720-724. The icons 720-724 include distinct shapes corresponding to respective statuses. For example, a circle shape may indicate that the user is available to participate in a wellness experience and a cross shape may indicate that the user is unavailable to participate in a wellness experience. The user may interact with the map by swiping their finger(s) along the screen of the client device 140 over the map to change the location displayed or change the magnification level of the map displayed. As the user interacts with the map, the group coordination engine 328 may use the GPS coordinates of a bounding box corresponding to the map visible to the user to determine which users are within the bounding box. The client device 140 may provide these GPS coordinates of the bounding box to the media server 130. The group coordination engine 328 may then display icons of the users within the map (e.g., the group coordination engine 328 provides the information of the icons to display to the client device 140).

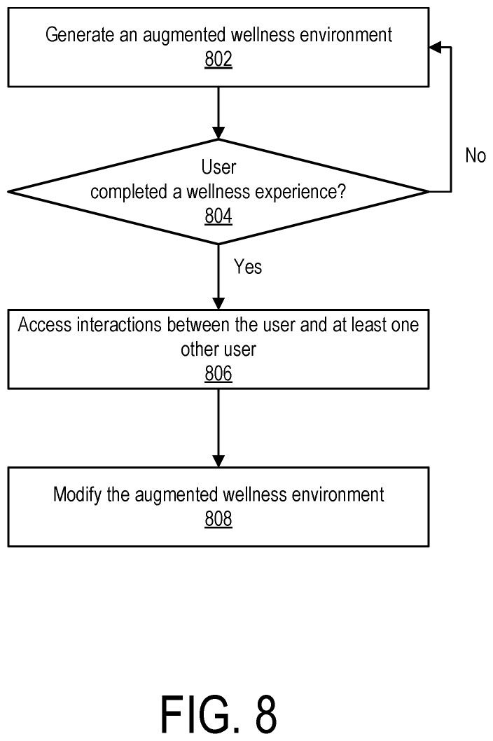

FIG. 8 illustrates an example embodiment of a process for generating an interactive wellness experience. In particular, the process includes modifying a wellness experience based on interactions between users. The process may be performed by the media server 130. The process may include additional, fewer, or different operations than shown in FIG. 8. The media server 130 generates 802 an augmented wellness environment. The augmented wellness environment may include a virtual object (e.g., a VR or AR object), which may include the experience assets described herein. The augmented wellness environment may also be generated by outputting one or more audio signals (e.g., ambient music). The augmented wellness environment may be generated by the experience creation engine 326 at a media processing device (e.g., the media processing device 110).

The media server 130 may determine 804 whether a user (e.g., of the media processing device 100 at which the augmented wellness environment is generated) has completed a wellness experience. In some embodiments, the media server 130 may alternatively determine if the user is currently engaged in a wellness experience. The application server 322 of the media server 130 may determine if the user has completed or is currently engaged in a wellness experience. If the user has completed the wellness experience, the media server 130 may proceed to accessing 806 interactions between the user and at least one other user. Alternatively, if the user has not completed the wellness experience, the media server 130 may return to generating 802 an augmented wellness environment for the user until the user has completed the wellness experience.

The media server 130 may access 806 interactions between the user and at least one other user. The media server 130 may determine that the user and another user were engaged in a joint wellness experience, where the interactions between the user include the presence and characteristics of the joint wellness experience. Example characteristics include the participants, how long they participated, their individual and combined mood scores during the joint wellness experience, experience assets generated during the joint wellness experience, the location of the joint wellness experience, a time of day during which the joint wellness experience took place, biometric data of the participants, the types of client devices used by the participants, or any other suitable descriptor of the joint wellness experience. The application server 322 of the media server 130 may access 806 these interactions.

The media server 130 modifies 808 the augmented wellness environment based on the accessed 806 interactions. In a first example modification, the group coordination engine 328 may determine that a participant has joined the joint wellness experience and instruct the experience creation engine 326 to display an additional experience asset from the experience asset database 336. In a second example modification, the group coordination engine 328 determines that a participant has left the joint wellness experience and instructs the experience creation engine to remove an existing experience asset that was being displayed to the participants. In a third example modification, the group coordination engine 326 may determine a combined mood score for the participants in the joint wellness experience (e.g., a score of the participants before determining 804 that a user has completed a wellness experience, leaving the joint wellness experience) and modify the audio signal output at the speakers of the client devices of the remaining participants.

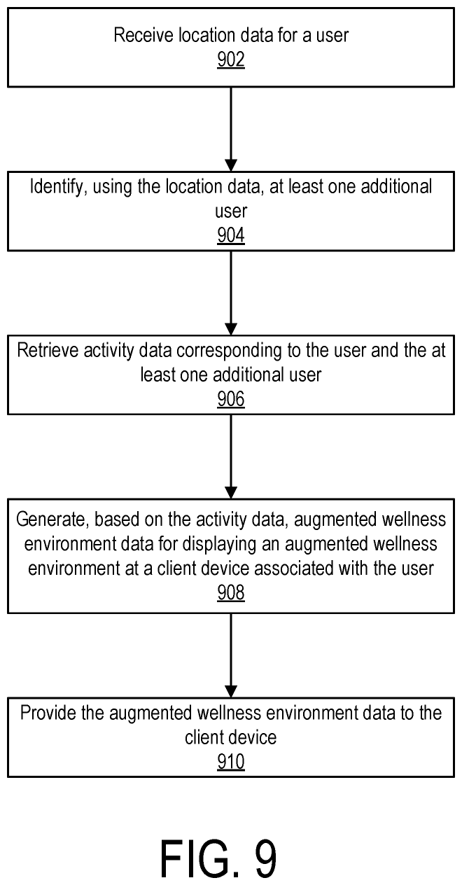

FIG. 9 illustrates an example embodiment of a process for providing augmented wellness environment data to a client device for displaying an augmented wellness environment. In particular, the process includes generating augmented wellness environment data based on activity data of multiple users. The process may be performed by the media server 130. The process may include additional, fewer, or different operations than shown in FIG. 9. The media server 130 retrieves 902 location data for a user. For example, the client device of the user may send GPS data to the media server 130. In one example, location data may include a social media status of a user that includes a location and time that the user is at the location. The media server 130 identifies 904, using the location data, at least one additional user. The group coordination engine 328 can identify, using location data received from client devices of various users within the same 100 meter radius, the various users. For example, the group coordination engine 328 identifies another user at the same park as the user of the client device using location data of the other user that indicates that they are also at the same park. The media server 130 retrieves 906 activity data corresponding to the user and the at least one additional user. The activity data can include biometric activity of the users. The activity data may also include data derived from the biometric activity of the users by the media server 130, such as a mood score.

The group coordination engine 328 may use, in addition to location data to determine how to modify the wellness activities for multiple users at the same location, the time at which users are participating in wellness activities. That is, the group coordination engine 328 may use a duration of time to limit how an augmented wellness environment may dynamically change. For example, the group coordination engine 328 may begin at the start of a day (e.g., beginning at midnight) and track users performing wellness activities at a particular location. After the day has ended, the group coordination engine 328 may begin anew, tracking users starting from midnight of the next day and generating the augmented wellness environments for users tracked during the next day. Although this example uses a daily augmented wellness environment generation cycle, durations of time that the group coordination engine 328 may use can be shorter (e.g., one hour or less) or longer (a week, month, etc.). The duration of time may be periodic (e.g., weekly, daily, hourly) or non-periodic (e.g., a Thanksgiving meditation event).

The media server 130 may generate 908, based on the activity data, augmented wellness environment data for displaying an augmented wellness environment data at the client device of the user or a media processing device of the user. The augmented wellness environment can include a virtual rendered object, which may also be referred to as a “virtual object.” In the previous example, the two users may have participated in a wellness activity at the same park. As one user joins the other in a wellness activity, which may be a joint wellness experience or an individual wellness experience, the media server 130 may generate 908 augmented wellness environment data that accounts for the activity data of the users who also participate in wellness activities at the park. The activity data may show that the users are decreasing their heart rates and breathing steadily, achieving a mood score that reflects a calmer state. The media server 130 may generate 908 augmented wellness environment data with more virtual assets as the users achieve a collective mood score that meet a target mood score (e.g., promoting calmness within a community of users). In one example, the activity data may show that a user who has performed a wellness activity within the park is stressed and thus contribute a mood score to the aggregate of users' activity data that penalizes the collective mood score of the community of users. In response, the media server 130 may generate augmented wellness environment data that removes virtual assets.

The media server 130 provides 910 the augmented wellness environment data to the user's client device. For example, the user may have an augmented reality experience generated at their smartphone. Alternatively or additionally, the media server 130 may provide 910 the augmented wellness environment data to a media processing device (e.g., for a virtual reality experience). The augmented wellness environment data takes into account the activity data of other users who are present or have been present around the same location as the user. Following the previous example, the AR experience generated at the smartphone includes AR objects that were generated with each user who had previously participated in a wellness activity in the park. Similarly, the user who is participating in the wellness activity themselves can also cause the media server 130 to generate an AR object to add to the augmented wellness environment due to the user's participation. An example of an augmented wellness environment generated at a client device or media processing device using augmented wellness environment data is shown in FIGS. 4 and 5.

In this way, the media server 130 may identify a particular location, retrieve activity data of the users who perform wellness activities at the location, and generate augmented wellness environment data based on the retrieved activity data for users presently and subsequently performing wellness activities at the location. The media server 130 is generating a virtual experience that does not isolate a user to their own experience. Rather, the media server 130 changes their virtual experience depending on other users' virtual experiences, creating a virtual experience that fosters community wellness.

### ADDITIONAL CONSIDERATIONS

Throughout this specification, some embodiments have used the expression “coupled” along with its derivatives. The term “coupled” as used herein is not necessarily limited to two or more elements being in direct physical or electrical contact. Rather, the term “coupled” may also encompass two or more elements that are not in direct contact with each other, but yet still co-operate or interact with each other.

Likewise, as used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Furthermore, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Upon reading this disclosure, those of skill in the art will appreciate still additional alternative structural and functional designs for the described embodiments as disclosed from the principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the scope defined in the appended claims.

What is claimed is:

1. A non-transitory computer-readable medium comprising instructions for generating an interactive wellness experience, the instructions, when executed by a computing system, causing the computing system to perform operations including:
receiving location data for a user;
identifying, using the location data, at least one additional user;
retrieving activity data corresponding to the user and the at least one additional user;
generating, based on the activity data, augmented wellness environment data for displaying an augmented wellness environment at a client device associated with the user, the augmented wellness environment including a virtual rendered object; and
providing the augmented wellness environment data to the client device.

2. The non-transitory computer readable medium of claim 1, wherein the operations further include:
determining respective mood scores of the user and the at least one additional user based on biometric data of the user and the at least one additional user;
generating updated augmented wellness environment data based on the mood scores; and
providing the updated augmented wellness environment data to the client device.

3. The non-transitory computer readable medium of claim 2, wherein the augmented wellness environment comprises output of a first audio signal using a speaker of the client device, and generating the updated augmented wellness environment data includes determining a second audio signal using the determined mood scores, the second audio signal being output by the speaker of the client device.

4. The non-transitory computer readable medium of claim 2, wherein the augmented wellness environment comprises display of the virtual object in a first color, and generating the updated augmented wellness environment data comprises:
determining a second color based on the determined mood scores; and
including, in the updated augmented wellness environment data, an instruction to display the virtual object in the second color.

5. The non-transitory computer readable medium of claim 1, wherein the operations further include:
accessing, responsive to detecting a user has completed a wellness experience, interactions between the user and the at least one additional user; and
modifying the augmented wellness environment based on the interactions.

6. The non-transitory computer readable medium of claim 5, wherein the interactions between the user and the at least one additional user comprises a joint wellness experience.

7. The non-transitory computer readable medium of claim 6, wherein the operations further include:
determining, during the joint wellness experience, that a number of participants in the joint wellness experience has changed, wherein the number of participants of the joint wellness experience changes as users begin or end respective wellness experiences within a predetermined distance of the participants of the joint wellness experience.

8. The non-transitory computer readable medium of claim 7, wherein generating the updated augmented wellness environment data comprises:
selecting, responsive to determining a number of participants of the joint wellness experience has increased, an additional virtual object; or
selecting, responsive to determining the number of participants of the joint wellness experience has decreased, a presently displayed virtual object to be removed from display.

9. The non-transitory computer readable medium of claim 6, wherein the updated augmented wellness environment data are generated based on at least one of:
a location at which the joint wellness experience is performed, the location tracked by a global positioning system (GPS) sensor of the client device, the client device communicatively coupled to the media processing device,
a duration of time during which the joint wellness experience is performed,
a time of day at which the joint wellness experience begins, or
biometric data of participants of the joint wellness experience, the biometric data monitored by one or more sensors coupled to respective media processing devices of the participants.

10. The non-transitory computer readable medium of claim 9, wherein the biometric data includes one or more of a heart rate or breathing rate.

11. The non-transitory computer readable medium of claim 1, wherein the virtual object is an avatar of the user, to the operations further comprising:
determining a number of wellness experiences that the user has completed; and
modifying the avatar based on the determined number of wellness experiences.

12. The non-transitory computer readable medium of claim 1, wherein the virtual object is an augmented reality object.

13. The non-transitory computer readable medium of claim 1, wherein the operations further include generating, for display at the client device, a dynamic map including icons corresponding to other users within a predetermined distance of the user, the icons indicating whether the other users are participating in a wellness experience.

14. A system for generating an interactive wellness experience, the system comprising:
an group coordination engine configured to:
receive location data for a user;
identify, using the location data, at least one additional user; and
retrieve activity data corresponding to the user and the at least one additional user;
an experience creation engine communicatively coupled to the application server, the experience creation engine configured to:
generate, based on the activity data, augmented wellness environment data for displaying an augmented wellness environment at a client device associated with the user, the augmented wellness environment including a virtual rendered object; and
provide the augmented wellness environment data to the client device.

15. The system of claim 14, wherein the experience creation engine is further configured to:
determine respective mood scores of the user and the at least one additional user based on biometric data of the user and the at least one additional user;
generate updated augmented wellness environment data based on the mood scores; and
provide the updated augmented wellness environment data to the client device.

16. The system of claim 15, wherein the experience creation engine is configured to:
generate the augmented wellness environment by:
outputting a first audio signal using a speaker of the client device; and
generate the updated augmented wellness environment data by:
determining a second audio signal using the determined mood scores, the second audio signal being output by the speaker of the client device.

17. The system of claim 15, wherein the experience creation engine is configured to:
generate the augmented wellness environment by:
generating for display the virtual object in a first color; and
generate the updated augmented wellness environment data by:
determining a second color based on the determined mood scores; and
including, in the updated augmented wellness environment data, an instruction to display the virtual object in the second color.

18. A method for generating an interactive wellness experience, the method comprising:
receiving location data for a user;
identifying, using the location data, at least one additional user;
retrieving activity data corresponding to the user and the at least one additional user;
generating, based on the activity data, augmented wellness environment data for displaying an augmented wellness environment at a client device associated with the user, the augmented wellness environment including a virtual rendered object; and
providing the augmented wellness environment data to the client device.

19. The method of claim 18, further comprising:
determining respective mood scores of the user and the at least one additional user based on biometric data of the user and the at least one additional user;
generating updated augmented wellness environment data based on the mood scores; and
providing the updated augmented wellness environment data to the client device.

20. The method of claim 19, wherein the augmented wellness environment comprises output of a first audio signal using a speaker of the client device, and generating the updated augmented wellness environment data includes determining a second audio signal using the determined mood scores, the second audio signal being output by the speaker of the client device.
